(19) United States
(12) Patent Application Publication
(10) Pub. No.: US 2020/0056027 A1
(43) Pub. Date: Feb. 20, 2020

(54) SOFT THERMOPLASTIC INJECTION MOLDED AND FLUSHABLE MATERIALS

(71) Applicant: KIMBERLY-CLARK WORLDWIDE, INC., Neenah, WI (US)

(72) Inventors: Alphonse DeMARCO, Seal Habour (CA); Gregory J. WIDEMAN, Menasha, WI (US); Austin N. PICKETT, Menasha, WI (US); Mark M. MLEZIVA, Appleton, WI (US)

(73) Assignee: Kimberly-Clark Worldwide, Inc., Neenah, WI (US)

(21) Appl. No.: 16/344,594

(22) PCT Filed: Oct. 30, 2017
(86) PCT No.: PCT/US2017/058992

§ 371 Date: Apr. 24, 2019

Related U.S. Application Data
(60) Provisional application No. 62/414,956, filed on Oct. 31, 2016.

(51) Int. Cl. C08L 23/08; C08L 29/04; C08L 75/04; C08L 53/02; A61F 13/26

(52) U.S. Cl. CPC C08L 23/0853; C08L 29/04; A61F 13/26; C08L 53/025; C08L 75/04

(57) ABSTRACT

A water-dispersible injection-moldable resin blend includes 20 wt. % to 80 wt. % thermoplastic elastomer, wherein the thermoplastic elastomer is ethylene-vinyl acetate (EVA), a thermoplastic polyurethane (TPU), or a styrenic block copolymer (SBC); and 80 wt. % to 20 wt. % modified poly(vinyl alcohol) (PVOH), wherein the modified PVOH is unmodified PVOH blended with glycerin. A flushable tampon applicator includes a grip region generally adjacent the outer end of the barrel, wherein the grip region of the barrel includes a water-dispersible injection-moldable resin blend including 20 wt. % to 80 wt. % thermoplastic elastomer, wherein the thermoplastic elastomer is ethylene-vinyl acetate (EVA), a thermoplastic polyurethane (TPU), or a styrenic block copolymer (SBC), and 80 wt. % to 20 wt. % modified poly(vinyl alcohol) (PVOH), wherein the modified PVOH is unmodified PVOH blended with glycerin.

### BACKGROUND

The present disclosure relates generally to tampon applicators. Vaginal tampons are disposable absorbent articles sized and shaped (e.g., cylindrical) for insertion into a women's vaginal canal for absorption of body fluids generally discharged during the woman's menstrual period. Insertion of the tampon into the vaginal canal is commonly achieved using a tampon applicator that comes initially assembled with the tampon.

Tampon applicators are typically of a two-piece construction, including a barrel in which the tampon is initially housed and a plunger moveable telescopically relative to the barrel to push the tampon out of the barrel and into the vaginal canal. The barrel has a tip that generally retains the tampon within the barrel until pushed through the tip by the plunger. In normal use, the applicator and more particularly the barrel of the applicator is held by the user by gripping one portion of the barrel (e.g., toward the trailing or plunger end of the barrel) and inserting the barrel, tip end first, into the vaginal canal. The barrel is pushed partially into the canal so that a portion (e.g., toward the leading or exit end of the tampon barrel) is disposed within the vaginal canal and is contact with the walls lining the canal. The plunger is then used to push the tampon out through the tip of the barrel and into the canal. The plunger and barrel are then removed from the vaginal canal, leaving the tampon in place.

In such use, the barrel (and plunger) of the applicator comes into contact with different body parts and/or skin regions of the user, such as the tip and part of the barrel contacting the walls lining the vaginal canal, while the user's finger(s) contact the barrel (and plunger) to grip and hold the barrel and to operate the plunger. The ability of the user to have a secure grip on the applicator while allowing the applicator to comfortably slide into and out of the vaginal canal is thus an important factor in acceptance of the applicator.

The applicator, which is often made of plastic or cardboard, is disposable. Thus, after the applicator has been used to insert the tampon into the user's vaginal canal the applicator is discarded. A convenient place to dispose of a tampon applicator is in a toilet bowl, even though all of the currently-used plastic applicators are ill-suited for such disposal. Consumers desire improvements to the cleanliness, discretion, and complexity of the tampon application and disposal process. In addition, wastewater treatment systems can benefit from the reduction or elimination of non-water-dispersible discards. Flushable feminine care products address both of these issues by providing consumers with discretion, cleanliness, and convenience benefits and by removing non-water-dispersible products from water treatment systems. To be considered flushable, however, the entire applicator should be flushable, including any grip material added to the applicator.

### SUMMARY

In one aspect, a water-dispersible injection-moldable resin blend includes 20 wt. % to 80 wt. % thermoplastic elastomer, wherein the thermoplastic elastomer is ethylene-vinyl acetate (EVA), a thermoplastic polyurethane (TPU), or a styrenic block copolymer (SBC); and 80 wt. % to 20 wt. % modified poly(vinyl alcohol) (PVOH), wherein the modified PVOH is unmodified PVOH blended with glycerin.

In another aspect, a flushable tampon applicator includes an elongated barrel having an interior chamber for housing a tampon therein, an outer end and an inner end spaced longitudinally from the outer end, a grip region generally adjacent the outer end of the barrel, a central region longitudinally adjacent the grip region and at least in part defining the interior chamber for housing the tampon within the barrel, and a barrel outer surface, wherein the barrel outer surface at the grip region of the barrel has a coefficient of friction that is greater than the barrel outer surface at the central region of the barrel. The tampon applicator also includes a plunger extending into the barrel at the outer end thereof and moveable relative to the barrel to expel the tampon from the barrel at the inner end of the barrel, wherein the grip region of the barrel includes a water-dispersible injection-moldable resin blend including 20 wt. % to 80 wt. % thermoplastic elastomer, wherein the thermoplastic elastomer is ethylene-vinyl acetate (EVA), a thermoplastic polyurethane (TPU), or a styrenic block copolymer (SBC), and 80 wt. % to 20 wt. % modified poly(vinyl alcohol) (PVOH), wherein the modified PVOH is unmodified PVOH blended with glycerin.

In an alternate aspect, a flushable tampon applicator includes an elongated barrel having an interior chamber for housing a tampon therein, an outer end and an inner end spaced longitudinally from the outer end, a grip region generally adjacent the outer end of the barrel, a central region longitudinally adjacent the grip region and at least in part defining the interior chamber for housing the tampon within the barrel, and a barrel outer surface, wherein the barrel outer surface at the grip region of the barrel has a coefficient of friction that is greater than the barrel outer surface at the central region of the barrel. The tampon applicator also includes a plunger extending into the barrel at the outer end thereof and moveable relative to the barrel to expel the tampon from the barrel at the inner end of the barrel; wherein the grip region of the barrel includes a water-dispersible injection-moldable resin blend including 20 wt. % to 60 wt. % poly(styrene-ethylene/butylene-styrene) (SEBS), and 80 wt. % to 40 wt. % modified poly(vinyl alcohol) (PVOH), wherein the modified PVOH is unmodified PVOH blended with glycerin.

Objects and advantages of the disclosure are set forth below in the following description, or can be learned through practice of the disclosure.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a perspective view of one aspect of a tampon applicator with a plunger of the applicator illustrated in an extended position relative to a barrel of the applicator and with a tip of the barrel open to illustrate construction of the barrel;

FIG. 2 is a longitudinal cross-section taken in the plane of line 2-2 of FIG. 1;

FIG. 3 is a perspective view of a second aspect of a barrel of a tampon applicator, with a tip of the barrel open to illustrate construction of the barrel;

FIG. 4 is a longitudinal cross-section taken in the plane of line 4-4 of FIG. 3;

FIG. 5 is a diagrammatic illustration of a basic injection molding machine; and

FIG. 6 is a diagrammatic illustration of an ASTM test specimen mold.

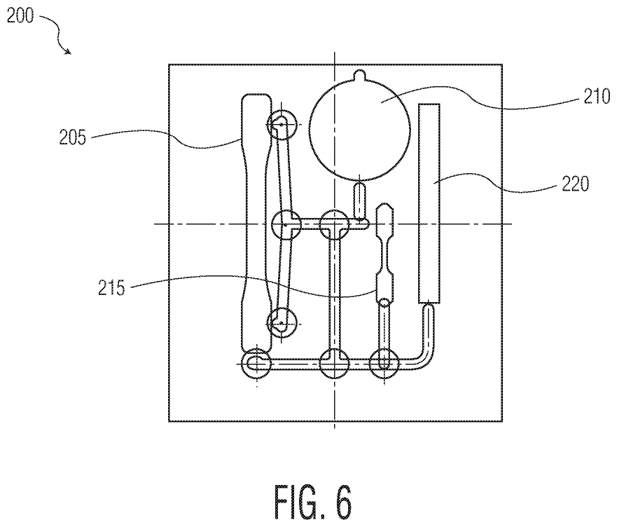

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

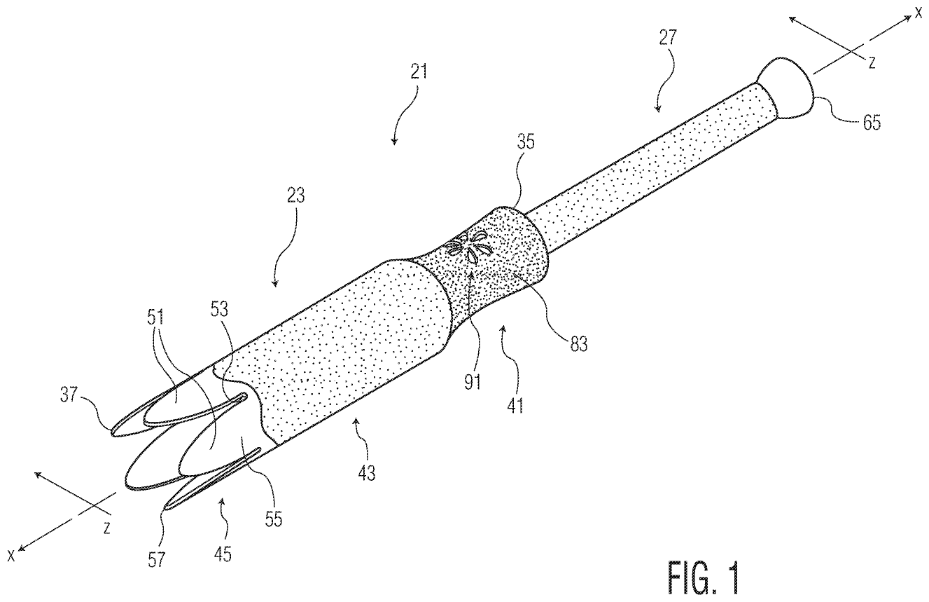

Referring now to the drawings and in particular to FIG. 1, one aspect of a tampon applicator is generally designated by reference numeral 21. The tampon applicator includes a barrel, indicated generally at 23, housing a tampon (not shown), and a plunger, indicated generally at 27, moveable telescopically relative to the barrel to expel the tampon from the barrel. In the various aspects herein the tampon applicator 21 is illustrated and described in connection with a vaginal tampon, i.e., a tampon such as a fibrous body sized and shaped (typically cylindrically shaped) for insertion into the vaginal canal of a female user to absorb menses, blood and other bodily fluids. It is understood, however, that the tampon applicator 21 can be used in connection with other suitable types of tampons. The tampon includes a withdrawal string (not shown) fastened to the tampon generally adjacent an outer or trailing end 31 thereof for use in pulling the tampon from the vaginal canal. Suitable tampon and withdrawal string materials and constructions are known to those skilled in the art and are not further described herein except to the extent necessary set forth the present disclosure.

The tampon applicator 21 has a longitudinal axis X, with the barrel 23 and plunger 27 being in coaxial relationship with each other on this axis. The plunger 27 is thus moveable telescopically along the longitudinal axis X from an extended position as illustrated in FIG. 1 to a delivery position (not shown) to expel the tampon from the barrel 23 of the applicator 21. It is understood, however, that the plunger 27 need not be coaxial with the barrel 23 and or the longitudinal axis X of the applicator 21 to remain within the scope of this disclosure.

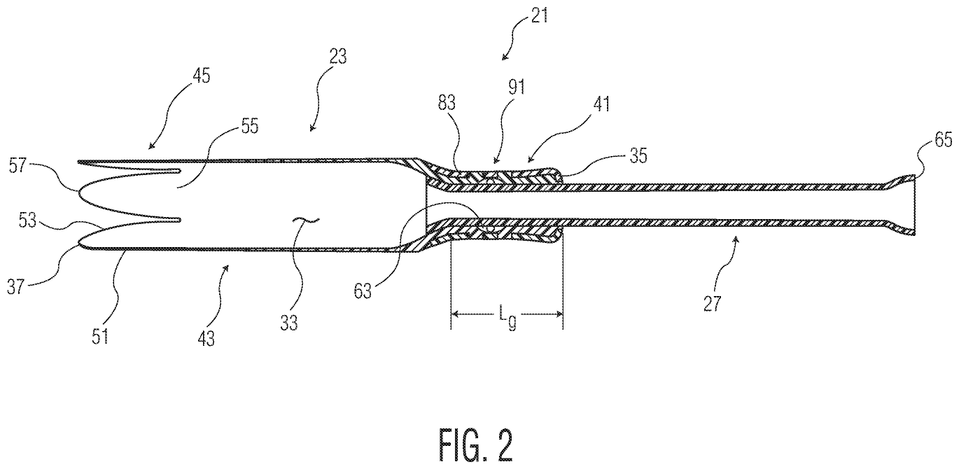

The barrel 23 of the tampon applicator 21 is suitably sized and shaped for housing the tampon within an interior chamber 33 (FIG. 2) of the barrel and for inserting the barrel into a body cavity of a user, such as the vaginal canal of a female user where the tampon is a vaginal tampon. The barrel 23 is generally elongated and also generally cylindrical, having an outer end 35, an inner end 37. The barrel 23 also broadly includes a grip region 41 adjacent the outer end of the barrel, an intermediate or central region 43 longitudinally adjacent the grip region and at least in part defining the interior chamber 33 housing the tampon, and an exit or tip region 45 longitudinally adjacent the central region in longitudinally spaced relationship with the grip region. The terms inner end and outer end as used herein are referenced relative to the orientation of the tampon applicator 21 and its various components during use thereof, with the barrel 23 being inserted, inner end 37 first, into the body cavity (e.g., the vaginal canal).

The tip region 45 of the barrel 23 includes a plurality of extensions, or what is commonly referred to as petals 51, separated by longitudinal slots 53. Each of the petals 51 extends longitudinally from a base 55 of the petal 51, where the petal is connected to and is more suitably formed integrally with the rest of the barrel 23, to a free end or tip 57 of the petal. More suitably, the width of each petal tapers inward from its base 55 toward its tip 57. The petals 51 are suitably configured in this manner to permit the petals to be bent inward during manufacture of the applicator 21 to generally close the barrel 23 at its inner end 37 to substantially enclose the tampon in the interior chamber 33 of the barrel during packaging and storage (e.g., prior to use). The slots 53 allow for bending of the petals 51 into their closed configuration during manufacture, and for flexing or bending transversely (e.g., radially in the illustrated aspect) outward upon application of force by the tampon when the tampon is guided out of the barrel 23 by the plunger 27.

With reference back to FIG. 2, the barrel 23 has an inner diameter (broadly, an inner cross-sectional dimension in the illustrated aspect) adjacent the outer end 35 (e.g., at and/or adjacent the grip region 41) of the barrel 23. This inner diameter is substantially less than that along the central region 43 of the barrel 23 (i.e., the portion that at least in part defines the interior chamber 33 in which the tampon is housed). This reduced diameter segment of the barrel 23 broadly defines a longitudinal guide channel 63 through which the plunger 27 extends and is supported by the barrel in coaxial (or at least longitudinal) relationship with the barrel. In particular, the guide channel has an inner diameter sized for sliding friction fit with the plunger.

In one particularly suitable aspect, the guide channel 63 has a length L_{g} sufficient to stably retain the plunger 27 coaxial with the barrel 23, i.e., to inhibit skewing of the plunger relative to the barrel as the plunger is pushed into the interior chamber 33 of the barrel to expel the tampon. For example, the length L_{g} of the guide channel 63 can suitably be in the range of about 5 mm to about 25 mm, more suitably in the range of about 12 mm to about 22 mm, and even more suitably about 15 mm to about 20 mm. As another example, the guide channel 63 of the applicator barrel 23 illustrated in FIG. 2 is approximately 18.6 mm in length. The length L_{g} of the guide channel 63, as used herein, refers to the longitudinal distance between the longitudinally innermost and outermost locations at which the inner diameter of the barrel 23 is sized for a close (e.g., relatively tight) fit and more suitably sliding friction contact with the plunger 27. Thus, it will be understood that the inner diameter of the barrel 23 can be substantially constant along the length L_{g} of the guide channel 63 as illustrated in FIG. 2, or the inner diameter can be sized approximately the same as an outer diameter of the plunger 27 at least at two longitudinally spaced locations, with the longitudinal spacing defining the length of the guide channel.

The plunger 27 is elongated and in the illustrated aspect is suitably hollow (FIGS. 1 and 2) so that the withdrawal string attached to the tampon can extend out through an outer end 65 of the plunger. It is understood though that the plunger 27 need not be hollow, and that the withdrawal string can extend other than through the plunger without departing from the scope of this disclosure. A substantial length of the plunger 27, extending to the outer end 65 thereof, is accessible exterior of the barrel 23 in the extended position of the plunger for gripping by the user to move the plunger relative the barrel. The plunger can have an increased outer diameter adjacent its outer end 65, such as in the form of a flange, ring, or bell shape as in the illustrated aspect or other suitable shape to facilitate gripping the plunger and to act as a stop to inhibit the outer end of the plunger against entering the barrel 23.

Suitable materials for use in constructing the barrel 23 and the plunger 27 are described in granted flushable tampon applicator patent documents including U.S. Pat. Nos. 9,320,656; 9,339,580; and 9,456,931, the contents of which are incorporated herein by reference to the extent they do not conflict herewith.

In accordance with one aspect, the barrel 23 is constructed such that the outer surface of the barrel at least at the central region 43 thereof, and more suitably at both the central region and the tip region 45 of the barrel, has a relatively low coefficient of friction to facilitate comfortable insertion of the barrel into the vaginal canal and removal therefrom. The barrel 23 is additionally constructed such that the outer surface of the barrel at its grip region 41 has a coefficient of friction that is substantially greater than the coefficient of friction at the central region 43 and tip region 45 of the barrel to facilitate gripping of the barrel while still providing a comfortable engagement between the outer surface of the barrel and the vaginal canal. Still more suitably, the barrel 23 is constructed to have a relatively soft feel and appearance while also providing the coefficient of friction differential between the grip region 41 and the central and tip regions 43, 45 of the barrel.

As one example, the barrel 23 according to one aspect can be suitably constructed of at least two materials that differ in at least one characteristic. More suitably, in one aspect the barrel is constructed of a first material that includes the tip region 45, central region 43 and an underlying portion of the grip region 41, and a second material that includes the overlying portion of the grip region. For example, the barrel 23 can be constructed along its full length (i.e., at the tip region 45, central region 43 and grip region 41) of a polymeric first or core layer 81 including a polyolefin such as, without limitation, polypropylene, polyethylene, low density polyethylene, high density polyethylene, linear low density polyethylene, near low density polyethylene, polyethylene terephthalate PET), nylon, polystyrene, polyvinyl chloride, polymethyl methacrylate, polyolefin elastomer, copolymers of alpha-olefins, and combinations thereof. More suitably the first or core layer of the barrel 23 is formed of a low density polyethylene or a polymeric blend that includes low density polyethylene, such as a combination of low density polyethylene and at least one of linear low density polyethylene or a high density polyethylene.

One or more additives can be added to the polymeric first layer of the barrel 23 (prior to molding) to enhance the slip characteristic (e.g., to provide a low coefficient of friction) of the barrel outer surface at least at the central region 43 of the barrel and more suitably at the central region and tip region 45 of the barrel. For example, suitable such additives include without limitation erucamide, dimethicone, oleamide, fatty acid amide and combinations thereof. It is understood that other additives can used to provide enhanced slip characteristics to the barrel 23 outer surface without departing from the scope of this disclosure. In other aspects the barrel 23 can instead, or additionally, be coated with a friction reducing, or slip agent such as wax, polyethylene, silicone, cellophane, clay and combinations thereof. In still other suitable aspects the barrel 23 can include a polymer blend melted together and co-extruded to provide a low coefficient of friction.

In other aspects, the tip region 45 of the barrel 23 can instead, or additionally be coated with a friction reducing agent so that the outer surface of the barrel at the tip region has a lower coefficient of friction than that of the central region of the barrel. Providing a surface roughness differential between the tip region 45 and the central region 43 also serves as a visual indicator of the reduced friction coefficient at the tip region.

The grip region 41 is suitably constructed of a second or skin layer 83 applied over the first or core layer along a longitudinal segment of the barrel 23 generally at the grip region thereof. Prior applications have used a thermoplastic elastomer (TPE) to provide the grip region with a soft, relatively rubbery feel that has a higher coefficient of friction than the first, or core layer that defines the outer surface of at least the central region 43 of the barrel 23. In these cases, injection molded articles are often over molded with a thermoplastic elastomer that provides a soft feeling aesthetic and/or grip area on the article or product. These thermoplastic elastomers are not water soluble, water dispersible, or flushable. As a result, they cannot be used on flushable articles such as a flushable tampon applicator.

A flushable applicator that can be injection molded and that has a consumer-appealing soft grip over-molded on the outer applicator tube is needed. To accomplish this, however, a flushable applicator needs a flushable grip material with properties similar to current TPEs. A water-dispersible grip material with a sufficiently-high coefficient of friction is disclosed herein.

A water-dispersible injection-moldable resin based on poly(vinyl alcohol) (PVOH) has been developed and is being used as the primary resin for injection molding of outer and inner (plunger) tubes in current tampon applicators. PVOH, however, is a relatively stiff resin unless significant plasticizer is added. As a result, when used as a grip material, the PVOH does not give the softness associated with the TPE resin currently employed for this purpose.

Previous developments of unmodified polyvinyl alcohol (PVOH) mixed with thermoplastic elastomers have been made into film and fiber structures that provide the desired characteristics such as enhanced ductility, enhanced softness, and lower noise generation. For these mixes to be water dispersible, however, the PVOH must be the majority volumetric component. Blending soft elastomers such as polyolefins and polyurethanes with PVOH can give the required softness and grip properties but the necessary level of soft elastomers makes the blends non-flushable.

It was discovered that ethylene-vinyl acetate (EVA) and to a greater extent styrenic block copolymers (SBCs) can provide the softness and grip properties at inclusion levels of 40-60% and still disperse in cold water in less than 3 hours as tested using a modified slosh box test.

SBCs such as poly(styrene-isoprene-styrene) (SIS), poly(styrene-butadiene-styrene) (SBS), poly(styrene-ethylene/butylene-styrene) (SEBS), poly(styrene-ethylene-propylene-styrene) (SEPS), poly(styrene-ethylene/propylene) (SEP), poly(styrene-b-isoprene/butadiene-b-styrene) (SEEPS) at a level of, for example, 60% and 40% in PVOH can disperse in cold water in under 30 minutes as outlined in Guidance Document for Assessing the Flushability of Nonwoven Consumer Products (INDA and EDANA, 2006); Test FG 522.2 Tier 2—Slosh Box Disintegration Test. These blends provide softness and grip comparable to the current TPE grip material. Without committing to an explanation, it is believed that, for example, the SEBS and PVOH resins are equally dispersed in the combination rather than forming separate domains. As such, as the PVOH disperses, the SEBS particles become dispersed as well rather than remaining as a complete layer.

Various elastomers blended with modified PVOH (containing the plasticizer glycerin) showed increased softness. Several candidates were found to meet flushability guidelines and have enough softness and tactility to be an acceptable replacement for a TPE grip on a flushable tampon applicator. Several SBC-grade blends were found to be flushable, even as the major component in a modified PVOH/SBC blend. These SBCs include SEBS, SEPS, and SBS.

Selecting the appropriate mix of materials yields a water-dispersible injection moldable resin that provides certain tactile properties desired for use as an overlay grip on a flushable tampon applicator. The resin is a blend of PVOH with a thermoplastic elastomer such as EVA, TPU, or an SBC. More specifically, an advantageous blend is PVOH with a specific SBC, SEBS. Other suitable SBCs include SIS, SBS, and SEPS. Also included in the blend is a plasticizer to modify the PVOH. Suitable plasticizers include glycerin.

The water-dispersible injection moldable resin blend includes a thermoplastic elastomer such as EVA, TPU, or SBC in an amount from 20 wt. % to 80 wt. %, in an amount from 20 wt. % to 60 wt. %, or in an amount from 20 wt. % to 50 wt. %. The water-dispersible injection moldable resin blend also includes modified PVOH in an amount from 20 wt. % to 80 wt. %, in an amount from 40 wt. % to 80 wt. %, or in an amount from 50 wt. % to 80 wt. %. The modified PVOH is preferably a blend of 60 to 85 wt. % unmodified PVOH, 5 to 20 wt. % glycerin, and optionally 3 to 5 wt. % colorant and/or slip additives.

In other aspects, the central region 43 and the grip region 41 can also be of different colors, which as used herein includes different hues as well as different shades of the same color as long as the different colors are visually perceptible by a human adult having 20/20 vision. Such a color change provides a visual cue to the user of a characteristic difference between the central region 43 and the tip region 45.

In the illustrated aspect of FIG. 1, a visual indicator, indicated generally at 91, is provided at the grip region 41 to facilitate identification by the user of the grip region location. The visual indicator 91 in FIG. 1, for example, includes a flower pattern formed in the grip region 41 to identify the grip region.

It is understood that the visual indicator 91 can be formed other than integrally with the barrel 23 during initial molding of the barrel, such as by imprinting the visual indicator on the barrel at the grip region (e.g., a textual message or a suitable image) without departing from the scope of this disclosure. It is also understood that a visual indicator 91 (other than the different material and/or color of the second or skin layer 83) can be omitted from the grip region 41.

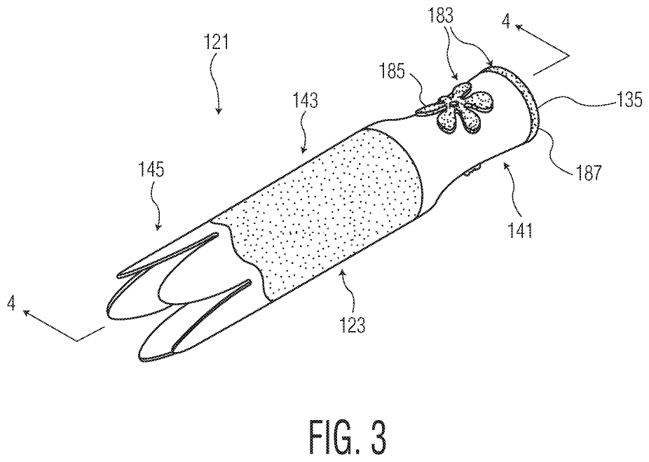

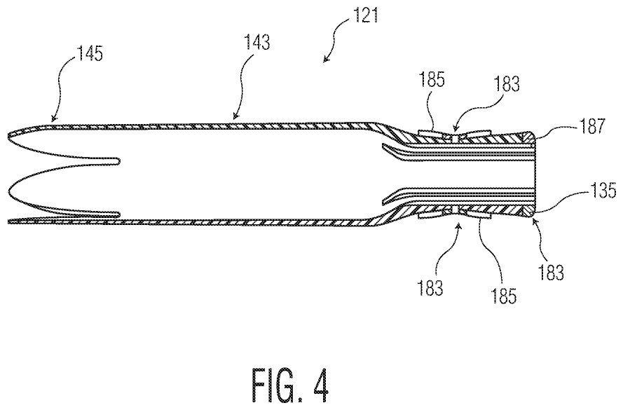

FIGS. 3 and 4 illustrate a barrel 123 of a second aspect of a tampon applicator 121. In this second aspect, the central region 143 and tip region 145 of the barrel are substantially the same as that of the aspect of FIGS. 1 and 2. At the grip region 141 of the barrel 123 of this second aspect the second or skin layer 183 itself is in the form of a raised flower pattern 185 (or other suitable pattern) and/or as a ring or collar 187 adjacent the outer end 135 of the barrel. A corresponding pattern is recessed into the outer surface of the first or core layer during molding and the patterned second or skin layer 183 is applied to the first or core layer to generally seat in the recessed pattern and extend transversely outward of the first layer to provide a higher coefficient of friction layer at the grip region 141. In this manner, the second or skin layer 183 in the form of a flower pattern also acts as a visual indicator of the grip region 141 location. It is understood that the second or skin layer 183 can be any suitable pattern other than a flower pattern, or other suitable raised surface, without departing from the scope of this disclosure.

### EXAMPLES

### Materials:

Poly(vinyl alcohol) (PVOH)—available from Sekisui, Dallas, Tex.
Selvol 502—PVOH partially hydrolyzed to 87%-89%; viscosity 3.0-3.7 cps

### Glycerin—Emery Cognis 916—Cognis Corporation, Cincinnati, Ohio

### Colorant/Slip—SCC85283—Standridge Color Corp., Social Circle, Ga.

Alternative polymer resins:
DYNAFLEX G6713 thermoplastic elastomer (TPE) from PolyOne, Avon Lake, Ohio
ECOFLEX C1200 biodegradable polyester from BASF, Germany
VISTAMAXX 2120 and 6102 polypropylene-based elastomer from Exxon Mobile, Houston, Tex.
ESTANE 2103 thermoplastic polyurethane (TPU) from Lubrizol, Cleveland, Ohio
ESCORENE Ultra EVA Copolymer 755.12 ethylene vinyl acetate from Exxon Mobile, Houston, Tex.
KRATON D1102 (SBS), G1637 (SEBS), and MD6716 (SEBS) styrenic block copolymers from Kraton Polymers, Houston, Tex.
SEPTON 1001 (SEP), 2004 (SEPS), 2063 (SEPS), 4033 (SEEPS), 8007 (SEBS) styrenic block copolymers from Kuraray America, Houston, Tex.

Resin Compounding: In general, formulated resins were produced using a ZSK-30 co-rotating twin screw extruder with 7 heated sections and a resin-compounding screw design. Resins were produced at a rate of 20 pounds per hour. PVOH, alternative resins, and a colorant/slip agent were feed through the main feed section through separate feeders. Glycerin was injected in section 3 of the extruder. The temperature profile per section, beginning at the main feed section, was 90°, 130°, 160°, 190°, 190°, 180°, and 145° C. The melt pressure ranged between 30-50 psi with the extruder torque of between 35 to 45%. The extruded polymer was uniform in color and flowed well from the die. The strands were air cooled and pelletized.

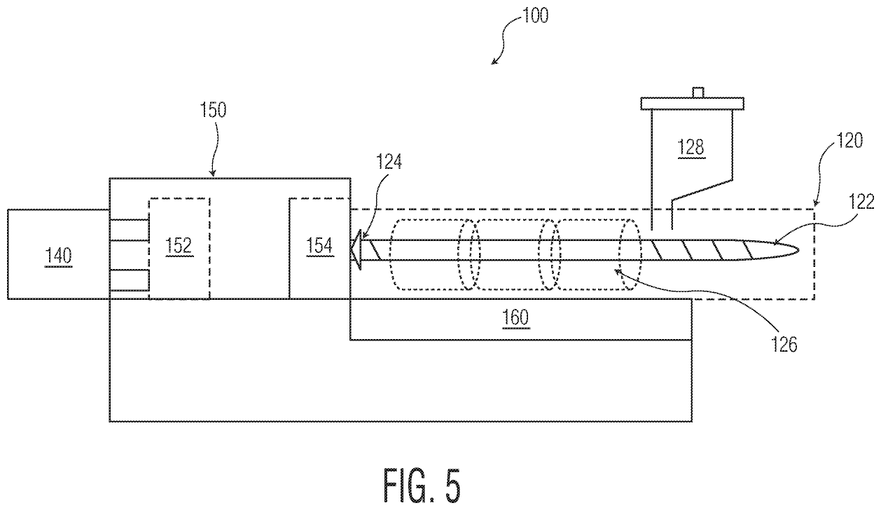

Injection Molding: The examples where processed on the Boy Machine 22D Injection Molder. This model has a 24.2 ton clamping force unit, a 24 mm plasticizing unit, and a shot size of 34 grams. FIG. 5 is a schematic of a basic injection molding machine 100. It shows the main components: the injection unit 120, the clamping unit 140, and the control panel 160. The injection molding cycle begins when the mold 150 closes, pairing the moveable platen 152 with the fixed platen 154. At this point, the screw 122 moves forward and injects the material through the nozzle 124 into the sprue, and the material fills the mold 150 (runners, gates, and cavities). During the packing phase, additional material is packed into the cavities. The material is cooled and solidifies in the mold while the screw 122 rotates counterclockwise backward, melting the plastic for the next shot using heating bands 126. New material is supplied by the hopper 128. The mold 150 opens and the parts are ejected. The next cycle begins when the mold 150 closes again.

The mold 200 used to produce specimens was an ASTM D638 standard test specimen mold from Master Precision Products, Inc., as illustrated in FIG. 3. This mold 200 contains a Tensile Type I specimen 205, a round disk 210, a Tensile Type V specimen 215, and an Izod bar 220.

Melt Flow Index: ASTM D1238 was used to determine the melt flow rate of the formulated thermoplastic resins.

Physical Properties: ASTM D638 was used to determine the physical properties of the injection molded parts.

Compression Force: Compression force is measured by placing an Izod bar specimen on a flat plate. A 6 mm diameter flat ended probe is advanced at a speed of 0.5 inch/min to contact the test piece. The probe is extended and the distance extended is recorded at various loads.

Flushability Assessment: Disintegration testing was performed as outlined in Guidance Document for Assessing the Flushability of Nonwoven Consumer Products (INDA and EDANA, 2006); Test FG 522.2 Tier 2—Slosh Box Disintegration Test. A round disc of each test resin is weighed and placed in 2L of water maintained at 15° C. and agitated at 25-26 cycles per minute. The time for the material to disperse completely and pass through a 1 mm screen is recorded. After a maximum of 180 minutes, the test is stopped, any remaining pieces larger than 1 mm are collected, dried, and weighed. The percent weight remaining of the disc is recorded. Under this test, a material is considered flushable if less than 5% of the material is retained by a 1 mm screen after 180 minutes.

### Example 1

Initial attempts at formulating a water-dispersible grip material sought to modify the current grip material, TPE. The current grip material, DYNAFLEX TPE, was blended with the modified PVOH used in flushable tampon applicators as described above. The material was compounded on the twin screw ZSK-30 extruder, pelletized, and injection molded into ASTM test pieces for analysis with the results listed in Table 1.

TABLE 1
Mixtures of PVOH and TPE

| | Slosh Box | Percent |
|---|---|---|
| | Time | Remaining |
| Description | minutes | % |
| 100% Dynaflex TPE Control | 180 | 100.0 |
| 95% Selvol 502 (14% Gly) - 5% Dynaflex | 45-60 | 0 |
| 90% Selvol 502(11% Gly) - 10% Dynaflex | 45-60 | 0 |
| 85% Selvol 502 (14% Gly) - 15% Dynaflex | 45-60 | 0 |
| 80% Selvol 502 (11% Gly) - 20% Dynaflex | 45-60 | 0 |
| 70% Selvol 502 (11% Gly) - 30% Dynaflex | 180 | 0.6 |

The level of TPE could not be increased above 30% and remain flushable as determined using the modified slosh box test. Softness and physical properties of the PVOH/TPE blends did not approach those of the control TPE alone, as shown in Table 2. Increasing the level of glycerin (Gly) plasticizer does have an effect on lowering the modulus of the material, but extremely high levels would be needed to lower it to the softness of the control TPE.

TABLE 2
Properties of PVOH and TPE Blends

| | Com- | | | |
|---|---|---|---|---|
| | pression | | | |
| | Force | Physicals | | |
| | 750 gf | Peak | | |
| | Load | Stress | Elongation | Modulus |
| Description | mm | MPa | % | MPa |
| 100% Dynaflex TPE Control | 0.537 | 0 | 44 | 1 |
| 95% Selvol 502 | 0.080 | 9 | 35 | 98 |
| (14% Gly) - 5% Dynaflex | | | | |
| 90% Selvol 502 | 0.046 | 12 | 40 | 136 |
| (11% Gly) - 10% Dynaflex | | | | |
| 85% Selvol 502 | 0.043 | 8 | 39 | 85 |
| (14% Gly) - 15% Dynaflex | | | | |
| 80% Selvol 502 | 0.045 | 8 | 33 | 91 |
| (11% Gly) - 20% Dynaflex | | | | |
| 70% Selvol 502 | 0.061 | 6 | 92 | 75 |
| (11% Gly) - 30% Dynaflex | | | | |

### Example 2

Hypothesizing that increasing the thermoplastic elastomer content would soften the PVOH material, the next step was to melt process blends of three types of elastomer resins: ethylene vinyl acetate (EVA), thermoplastic polyurethane (TPU), and a styrenic block copolymer (SBC). All three types of resins processed well as shown in Table 3.

TABLE 3
Melt Processing Conditions

| | Melt Index | Tₘₑₗₜ | Pₘₑₗₜ | Torque |
|---|---|---|---|---|
| Description | g/10 min | (° C.) | (psi) | (%) |
| 83% Selvol 502 | 109 | 181 | 35 | 38 |
| (13% Gly) | | | | |
| 63% Selvol 502 | 58 | 175 | 65 | 40 |
| (13% Gly) - 20% TPU | | | | |
| 43% Selvol 502 | 49 | 175 | 80 | 44 |
| (13% Gly) - 40% TPU | | | | |
| 23% Selvol 502 | 40 | 175 | 90 | 41 |
| (13% Gly) - 60% TPU | | | | |
| 63% Selvol 502 | 53 | 182 | 50 | 34 |
| (13% Gly) - 20% EVA | | | | |
| 43% Selvol 502 | 69 | 182 | 40 | 28 |
| (13% Gly) - 40% EVA | | | | |
| 23% Selvol 502 | 78 | 181 | 30 | 23 |
| (13% Gly) - 60% EVA | | | | |
| 63% Selvol 502 | 67 | 177 | 50 | 40 |
| (13% Gly) - 20% SEBS | | | | |
| 43% Selvol 502 | 66 | 177 | 45 | 33 |
| (13% Gly) - 40% SEBS | | | | |
| 23% Selvol 502 | 63 | 176 | 35 | 29 |
| (13% Gly) - 60% SEBS | | | | |
| 16% Selvol 502 | 60 | 172 | 30 | 30 |
| (10% Gly) - 70% SEBS | | | | |

Following injection molding of the test resins, flushability testing demonstrated that the blends with the SEBS resin were flushable (see Table 4) even when SEBS was the majority resin, contrary to the teachings of the prior art.

TABLE 4
Flushability of Elastomer Blends

| | Slosh Box | Percent |
|---|---|---|
| | Time | Remaining |
| Description | minutes | % |
| 83% Selvol 502 | 40 | 0 |
| (13% Gly) | | |
| 63% Selvol 502 | 60 | 0 |
| (13% Gly) - 20% TPU | | |
| 43% Selvol 502 | 180 | 100.0 |
| (13% Gly) - 40% TPU | | |
| 23% Selvol 502 | 180 | 100.0 |
| (13% Gly) - 60% TPU | | |
| 63% Selvol 502 | 40 | 0 |
| (13% Gly) - 20% EVA | | |
| 43% Selvol 502 | 180 | 5.0 |
| (13% Gly) - 40% EVA | | |
| 23% Selvol 502 | 180 | 18.5 |
| (13% Gly) - 60% EVA | | |
| 63% Selvol 502 (13% Gly) - 20% | 30 | 0 |
| Kraton MD6716 SEBS | | |
| 43% Selvol 502 (13% Gly) - 40% | 30 | 0 |
| Kraton MD6716 SEBS | | |
| 23% Selvol 502 (13% Gly) - 60% | 30 | 0 |
| Kraton MD6716 SEBS | | |
| 16% Selvol 502 (10% Gly) - 70% | 180 | 14.3 |
| Kraton MD6716 SEBS | | |

Compression testing to demonstrate softness, as shown in Table 5, demonstrated that greater amounts of SEBS softened the PVOH blend, but the softness was still less than TPE alone. In Table 5, a higher compression distance at a given compression force means the material is softer.

TABLE 5
Compression Distance at Low Loads

| | Compression |
|---|---|
| | Distance |
| | 750 gf Load |
| Description | mm |
| 100% Dynaflex TPE | 0.537 |
| 83% Selvol 502 (13% Gly) | 0.032 |
| 63% Selvol 502 (13% Gly) - 20% TPU | 0.035 |
| 43% Selvol 502 (13% Gly) - 40% TPU | 0.048 |
| 23% Selvol 502 (13% Gly) - 60% TPU | 0.084 |
| 63% Selvol 502 (13% Gly) - 20% EVA | 0.036 |
| 43% Selvol 502 (13% Gly) - 40% EVA | 0.069 |
| 23% Selvol 502 (13% Gly) - 60% EVA | 0.117 |
| 63% Selvol 502 (13% Gly) - 20% SEBS | 0.042 |
| 43% Selvol 502 (13% Gly) - 40% SEBS | 0.054 |
| 23% Selvol 502 (13% Gly) - 60% SEBS | 0.093 |
| 16% Selvol 502 (10% Gly) - 70% SEBS | 0.109 |

When over-molded as a grip onto a current tampon applicator, the SEBS blend had a similar feel to that of the control TPE.

### Example 3

A survey of various SBC polymers was conducted to determine their overall processability, flushability, and physical properties. The SBCs tested were SEP, SEEPS, SEPS, SBS, and SEBS. Two of the SBC grades could not be processed above a 50% blend, as demonstrated in Table 6.

TABLE 6
Melt Processing PVOH/SBC Blends

| | Melt Index | Tₘₑₗₜ | Pₘₑₗₜ | Torque |
|---|---|---|---|---|
| Description | g/10 min | (° C.) | (psi) | (%) |
| 46% Selvol 502 (10% Gly) - 40% Septon 1001 SEP | 11 | 180 | 85 | 41 |
| 26% Selvol 502 (10% Gly) - 60% Septon 1001 SEP | 2 | 180 | 120 | 43 |
| 46% Selvol 502 (10% Gly) - 40% Septon 2004 SEPS | 48 | 179 | 45 | 37 |
| 26% Selvol 502 (10% Gly) - 60% Septon 2004 SEPS | 32 | 179 | 40 | 37 |
| 46% Selvol 502 (10% Gly) - 40% Septon 2063 SEPS | 44 | 179 | 50 | 39 |
| 26% Selvol 502 (10% Gly) - 60% Septon 2063 SEPS | 25 | 179 | 40 | 33 |
| 46% Selvol 502 (10% Gly) - 40% Septon 4033 SEEPS | 3 | 179 | 105 | 50 |
| 36% Selvol 502 (10% Gly) - 50% Septon 4033 SEEPS | 1 | 173 | 135 | 46 |
| 46% Selvol 502 (10% Gly) - 40% Kraton D1102 SBS | 49 | 175 | 40 | 39 |
| 26% Selvol 502 (10% Gly) - 60% Kraton D1102 SBS | 55 | 175 | 35 | 34 |
| 46% Selvol 502 (10% Gly) - 40% Septon 8007 SEBS | 9 | 174 | 105 | 50 |
| 36% Selvol 502 (10% Gly) - 50% Septon 8007 SEBS | 4 | 175 | 130 | 49 |
| 63% Selvol 502 (13% Gly) - 20% Kraton G1637 SEBS | 69 | 182 | 50 | 39 |
| 43% Selvol 502 (13% Gly) - 40% Kraton G1637 SEBS | 26 | 180 | 80 | 41 |
| 33% Selvol 502 (13% Gly) - 50% Kraton G1637 SEBS | 23 | 180 | 40 | 36 |

Several grades of SBCs were flushable as the major component as demonstrated in Table 7. The guideline currently states that to be considered flushable, less than 5% of the material is retained by a 1 mm screen after 180 minutes. The flushable materials include SEPS, SBS, and SEBS.

TABLE 7
Flushability of PVOH/SBC Blends

| | Slosh Box Time | Percent Remaining |
|---|---|---|
| Description | minutes | % |
| 46% Selvol 502 (10% Gly) - 40% Septon 1001 SEP | 30 | 0 |
| 26% Selvol 502 (10% Gly) - 60% Septon 1001 SEP | 180 | 41.0 |
| 46% Selvol 502 (10% Gly) - 40% Septon 2004 SEPS | 30 | 0 |
| 26% Selvol 502 (10% Gly) - 60% Septon 2004 SEPS | 180 | 11.6 |
| 46% Selvol 502 (10% Gly) - 40% Septon 2063 SEPS | 30 | 0 |
| 26% Selvol 502 (10% Gly) - 60% Septon 2063 SEPS | 30 | 0 |
| 46% Selvol 502 (10% Gly) - 40% Septon 4033 SEEPS | 60 | 0 |
| 36% Selvol 502 (10% Gly) - 50% Septon 4033 SEEPS | 180 | 13.0 |
| 46% Selvol 502 (10% Gly) - 40% Kraton D1102 SBS | 30 | 0 |
| 26% Selvol 502 (10% Gly) - 60% Kraton D1102 SBS | 180 | 2.3 |
| 46% Selvol 502 (10% Gly) - 40% Septon 8007 SEBS | 30 | 0 |
| 36% Selvol 502 (10% Gly) - 50% Septon 8007 SEBS | 180 | 1.4 |
| 63% Selvol 502 (13% Gly) - 20% Kraton G1637 SEBS | 30 | 0 |
| 43% Selvol 502 (13% Gly) - 40% Kraton G1637 SEBS | 25 | 0 |
| 33% Selvol 502 (13% Gly) - 50% Kraton G1637 SEBS | 180 | 2.4 |

Compression and physical properties testing of these blends also confirmed that the greater amount of SBC that can be incorporated into the PVOH blend, the softer the resulting material, as demonstrated in Table 8.

TABLE 8
Physical Properties of PVOH/SBC Blends

| | Compression | Physicals | | |
|---|---|---|---|---|
| | Distance | Peak | | |
| | 750 gf Load | Stress | Elongation | Modulus |
| Description | mm | MPa | % | MPa |
| 46% Selvol 502 (10% Gly) - 40% Septon 1001 SEP | 0.057 | 6 | 26 | 120 |
| 26% Selvol 502 (10% Gly) - 60% Septon 1001 SEP | 0.060 | 2 | 18 | 50 |
| 46% Selvol 502 (10% Gly) - 40% Septon 2004 SEPS | 0.053 | 7 | 80 | 107 |
| 26% Selvol 502 (10% Gly) - 60% Septon 2004 SEPS | 0.080 | 3 | 57 | 26 |
| 46% Selvol 502 (10% Gly) - 40% Septon 2063 SEPS | 0.043 | 6 | 47 | 81 |
| 26% Selvol 502 (10% Gly) - 60% Septon 2063 SEPS | 0.085 | 2 | 48 | 15 |
| 46% Selvol 502 (10% Gly) - 40% Septon 4033 SEEPS | 0.041 | 7 | 66 | 116 |
| 36% Selvol 502 (10% Gly) - 50% Septon 4033 SEEPS | 0.066 | 5 | 48 | 75 |
| 46% Selvol 502 (10% Gly) - 40% Kraton D1102 SBS | 0.045 | 8 | 115 | 130 |
| 26% Selvol 502 (10% Gly) - 60% Kraton D1102 SBS | 0.090 | 3 | 98 | 43 |
| 46% Selvol 502 (10% Gly) - 40% Septon 8007 SEBS | 0.055 | 7 | 54 | 115 |
| 36% Selvol 502 (10% Gly) - 50% Septon 8007 SEBS | 0.069 | 5 | 52 | 70 |
| 63% Selvol 502 (13% Gly) - 20% Kraton G1637 SEBS | 0.049 | 8 | 45 | 93 |
| 43% Selvol 502 (13% Gly) - 40% Kraton G1637 SEBS | 0.074 | 5 | 39 | 48 |
| 33% Selvol 502 (13% Gly) - 50% Kraton G1637 SEBS | 0.089 | 3 | 40 | 33 |

Although blending of various elastomers with modified PVOH did not result in a material as soft as TPE, several candidates were found to meet flushability guidelines and have softness sufficient to replace TPE as a grip material on a flushable tampon applicator. Several SBC grades were found to be flushable, even as the major component in a PVOH/SBC blend.

Blends with SEBS (KRATON G1637 (0.089)), SEPS (SEPTON 2063 (0.085)), and SBS (KRATON D1102 (0.090)) as the majority resin were flushable and had a compression distance at 750 gf load close to 20% of the control TPE (0.537). When applied as a grip on a tampon applicator, each material had similar softness and grip attributes to that of a TPE grip.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there can be additional elements other than the listed elements.

As various changes could be made in the above products without departing from the scope of the disclosure, it is intended that all matter contained in the above description and shown in the accompanying drawings shall be interpreted as illustrative and not in a limiting sense.

While the disclosure has been described in detail with respect to the specific aspects thereof, it will be appreciated that those skilled in the art, upon attaining an understanding of the foregoing, can readily conceive of alterations to, variations of, and equivalents to these aspects. Accordingly, the scope of the present disclosure should be assessed as that of the appended claims and any equivalents thereto.

What is claimed is:

1. A water-dispersible injection-moldable resin blend comprising:
20 wt. % to 80 wt. % thermoplastic elastomer, wherein the thermoplastic elastomer is ethylene-vinyl acetate (EVA), a thermoplastic polyurethane (TPU), or a styrenic block copolymer (SBC); and
80 wt. % to 20 wt. % modified poly(vinyl alcohol) (PVOH), wherein the modified PVOH is unmodified PVOH blended with glycerin.

2. The resin blend of claim 1, wherein the thermoplastic elastomer is an SBC.

3. The resin blend of claim 2, wherein the SBC is one of poly(styrene-isoprene-styrene) (SIS), poly(styrene-butadiene-styrene) (SBS), poly(styrene-ethylene/butylene-styrene) (SEBS), poly(styrene-ethylene-propylene-styrene) (SEPS), poly(styrene-ethylene/propylene) (SEP), and poly(styrene-b-isoprene/butadiene-b-styrene) (SEEPS).

4. The resin blend of claim 2, wherein the SBC is poly(styrene-ethylene/butylene-styrene) (SEBS).

5. The resin blend of claim 1, wherein the unmodified PVOH is partially hydrolyzed (87-89%) with a viscosity range of 3.0-3.7 cps.

6. The resin blend of claim 1, wherein the modified PVOH is a blend of 60 wt. % to 85 wt. % unmodified PVOH, 5 wt. % to 20 wt. % glycerin, and optionally 3 wt. % to 5 wt. % colorant and/or slip additives.

7. The resin blend of claim 1, wherein the resin blend includes from 20 wt. % to 60 wt. % thermoplastic elastomer and from 40 wt. % to 80 wt. % modified PVOH.

8. The resin blend of claim 1, wherein the resin blend includes from 20 wt. % to 50 wt. % thermoplastic elastomer and from 50 wt. % to 80 wt. % modified PVOH.

9. The resin blend of claim 1, wherein the resin blend is flushable according to Guidance Document for Assessing the Flushability of Nonwoven Consumer Products (INDA and EDANA, 2006); Test FG 522.2 Tier 2—Slosh Box Disintegration Test.

10. A flushable tampon applicator comprising:
an elongated barrel having
an interior chamber for housing a tampon therein,
an outer end and an inner end spaced longitudinally from the outer end,
a grip region generally adjacent the outer end of the barrel,
a central region longitudinally adjacent the grip region and at least in part defining the interior chamber for housing the tampon within the barrel, and
a barrel outer surface, wherein the barrel outer surface at the grip region of the barrel has a coefficient of friction that is greater than the barrel outer surface at the central region of the barrel; and
a plunger extending into the barrel at the outer end thereof and moveable relative to the barrel to expel the tampon from the barrel at the inner end of the barrel;
wherein the grip region of the barrel comprises a water-dispersible injection-moldable resin blend comprising 20 wt. % to 80 wt. % thermoplastic elastomer, wherein the thermoplastic elastomer is ethylene-vinyl acetate (EVA), a thermoplastic polyurethane (TPU), or a styrenic block copolymer (SBC), and 80 wt. % to 20 wt. % modified poly(vinyl alcohol) (PVOH), wherein the modified PVOH is unmodified PVOH blended with glycerin.

11. The tampon applicator of claim 10, wherein the thermoplastic elastomer is an SBC.

12. The tampon applicator of claim 11, wherein the SBC is one of poly(styrene-isoprene-styrene) (SIS), poly(styrene-butadiene-styrene) (SBS), poly(styrene-ethylene/butylene-styrene) (SEBS), poly(styrene-ethylene-propylene-styrene) (SEPS), poly(styrene-ethylene/propylene) (SEP), and poly(styrene-b-isoprene/butadiene-b-styrene) (SEEPS).

13. The tampon applicator of claim 11, wherein the SBC is poly(styrene-ethylene/butylene-styrene) (SEBS).

14. The tampon applicator of claim 10, wherein the unmodified PVOH is partially hydrolyzed (87-89%) with a viscosity range of 3.0-3.7 cps.

15. The tampon applicator of claim 10, wherein the modified PVOH is a blend of 60 wt. % to 85 wt. % unmodified PVOH, 5 wt. % to 20 wt. % glycerin, and optionally 3 wt. % to 5 wt. % colorant and/or slip additives.

16. The tampon applicator of claim 10, wherein the resin blend includes from 20 wt. % to 60 wt. % thermoplastic elastomer and from 40 wt. % to 80 wt. % modified PVOH.

17. The tampon applicator of claim 10, wherein the resin blend includes from 20 wt. % to 50 wt. % thermoplastic elastomer and from 50 wt. % to 80 wt. % modified PVOH.

18. The tampon applicator of claim 10, wherein the tampon applicator disperses in less than 180 minutes according to Guidance Document for Assessing the Flushability of Nonwoven Consumer Products (INDA and EDANA, 2006); Test FG 522.2 Tier 2—Slosh Box Disintegration Test.

19. A flushable tampon applicator comprising:
an elongated barrel having
an interior chamber for housing a tampon therein,
an outer end and an inner end spaced longitudinally from the outer end,
a grip region generally adjacent the outer end of the barrel,
a central region longitudinally adjacent the grip region and at least in part defining the interior chamber for housing the tampon within the barrel, and
a barrel outer surface, wherein the barrel outer surface at the grip region of the barrel has a coefficient of friction that is greater than the barrel outer surface at the central region of the barrel; and
a plunger extending into the barrel at the outer end thereof and moveable relative to the barrel to expel the tampon from the barrel at the inner end of the barrel;
wherein the grip region of the barrel comprises a water-dispersible injection-moldable resin blend comprising 20 wt. % to 60 wt. % poly(styrene-ethylene/butylene-styrene) (SEBS), and 80 wt. % to 40 wt. % modified poly(vinyl alcohol) (PVOH), wherein the modified PVOH is unmodified PVOH blended with glycerin.

20. The tampon applicator of claim 19, wherein the unmodified PVOH is partially hydrolyzed (87-89%) with a viscosity range of 3.0-3.7 cps.
